(19) United States
(12) Patent Application Publication
(10) Pub. No.: US 2008/0241231 A1
(43) Pub. Date: Oct. 2, 2008

(54) TRANSDERMAL DELIVERY OF DEXAMETHASONE AND PROMETHAZINE

(75) Inventors: Dave Gonyer, Cardiff by the Sea, CA (US); Matthew Heck, Encinitas, CA (US)

Correspondence Address: WILSON SONSINI GOODRICH & ROSATI, 650 PAGE MILL ROAD, PALO ALTO, CA, 94304-1050, US

(73) Assignees: Victory Pharma, Incorporated, San Diego, CA (US); Medgenex, Incorporated, San Diego, CA (US)

(21) Appl. No.: 12/035,349

(22) Filed: Feb. 21, 2008

Related U.S. Application Data
(60) Provisional application No. 60/890,988, filed on Feb. 21, 2007.

(51) Int. Cl. A61K 9/70; A61K 31/56; A61P 1/08

(52) U.S. Cl. 424/449; 514/180

(57) ABSTRACT

Relief or prophylaxis from nausea and/or emesis are provided by transdermal compositions comprising dexamethasone and promethazine. The transdermal compositions are in the form of transdermal solutions and/or suspensions, such as lotions, pastes, gels, ointments or transdermal patches.

### CROSS REFERENCE TO RELATED APPLICATIONS AND PRIORITY CLAIM

This application claims priority to U.S. Provisional patent application Ser. No. 60/890,988, filed Feb. 21, 2007, which is incorporated by reference herein in its entirety.

### FIELD OF THE INVENTION

The present invention relates to transdermal delivery of a combination of dexamethasone and promethazine as well as pharmaceutical compositions, manufactures and methods of using the same.

### BACKGROUND OF THE INVENTION

Nausea and vomiting are symptoms arising out of several different, unrelated physical conditions, many of which do not directly involve the stomach. Vomiting (emesis) is the forceful expulsion of stomach contents through the esophagus and mouth; nausea is the queasy feeling that one is going to vomit. There are various causes of nausea and vomiting. Motion sickness is a common form of nausea that may or may not include vomiting, which is caused by a difference in perception of motion between the inner ear and the eyes, often while riding some sort of mechanical conveyance, such as a car, an airplane or a boat. Morning sickness involves nausea and vomiting caused by hormonal fluctuations in pregnant women; it may be initiated or exacerbated by any of a variety of sensory cues, especially smells, some of which may actually seem pleasant to the same woman when she is not pregnant. Various drugs, other environmental stressors, migraine headaches, disease states and psychiatric conditions are also known to give rise to nausea and vomiting.

While vomiting may, in some circumstances, serve the purpose of expelling harmful substances from the stomach, prolonged, frequent or severe vomiting is generally undesirable or even life threatening. Likewise, nausea is an uncomfortable feeling that may be accompanied by sweating, excessive salivating, chills or other unpleasant symptoms. Especially in the case of motion sickness, the cause of nausea and vomiting may be nearly or entirely benign, while the effects may range in severity from mildly uncomfortable to debilitating. Even where vomiting serves an initially salutary purpose, e.g. to expel a toxin from the body, continued or especially violent vomiting may be undesirable or deleterious to the patient. Several anti-nausea medications (broadly referred to as antiemetic drugs, or simply antiemetics) have been developed; but there are still patients who do not respond to the available antiemetics or are sensitive to, or demonstrate poor compliance with, current anti-nausea therapies. There is thus a need for a new and improved antiemetic drug.

The invention meets the foregoing need and provides related advantages as well.

### SUMMARY

The invention meets the foregoing need, and provides further advantages as well, by providing a pharmaceutical composition for transdermal drug delivery, comprising dexamethasone and promethazine, or a pharmaceutically acceptable salt thereof. The composition is adapted to deliver a combination of dexamethasone and promethazine across the skin. In some embodiments, the composition provides a therapeutically effective dose of a combination of dexamethasone and promethazine. In some particular embodiments, the composition is presented in unit dosage form. In some embodiments, the composition comprises an anti-emetic and/or anti-nausea effective amount of the combination of dexamethasone and promethazine, or a pharmaceutically acceptable salt thereof. In some embodiments, the composition further comprises a skin penetration enhancer.

The foregoing and other needs are met by embodiments of the invention that provide a composition for transdermal drug delivery, comprising dexamethasone and promethazine, or a pharmaceutically acceptable salt thereof, in admixture with a pharmaceutically acceptable transdermal delivery vehicle. In some embodiments, the composition is in unit dosage form. In some particular embodiments, the composition comprises an anti-emetic and/or anti-nausea effective amount of the combination of dexamethasone and promethazine, or a pharmaceutically acceptable salt thereof. In some embodiments, the composition further comprises a skin penetration enhancer.

Further, the foregoing and other needs are met by embodiments of the invention that provide a composition for transdermal drug delivery, comprising dexamethasone and promethazine, or a pharmaceutically acceptable salt thereof, in admixture with a pharmaceutically acceptable transdermal delivery excipient, adjuvant or penetration enhancer. In some embodiments, the composition is in unit dosage form. In some particular embodiments, the composition comprises an anti-emetic and/or anti-nausea effective amount of the combination of dexamethasone and promethazine, or a pharmaceutically acceptable salt thereof. Furthermore, the invention provides a transdermal patch comprising a therapeutically effective amount of dexamethasone and promethazine, or a pharmaceutically acceptable salt thereof. In some embodiments, the patch is selected from a single layer medicine in adhesive patch, a multi-layer medicine in adhesive patch, a reservoir patch, a matrix patch, a microneedle patch or an iontophoretic patch.

The present invention also provides, in certain embodiments, a method of treating or preventing emesis in a subject, comprising transdermally administering to the subject a therapeutically effective amount of a combination of dexamethasone and promethazine or a pharmaceutically acceptable salt thereof. In some embodiments, the method comprises employing a therapeutically effective amount of dexamethasone and promethazine administered in admixture with a topically-applied vehicle. In other embodiments, the method comprises using a therapeutically effective amount of dexamethasone and promethazine administered as a transdermal patch. In still further embodiments, the transdermal patch is adapted for sustained release.

The foregoing and further advantages are met by embodiments of the invention, which provide a kit comprising an antiemetically effective combination of dexamethasone and promethazine for transdermal administration. In some embodiments, the kit further comprises instructions for use of the combination of dexamethasone and promethazine as an antiemetic. In some particular embodiments, the invention provides a kit comprising a first patch comprising dexamethasone. Where a single patch is comprised within the kit, the patch contains a therapeutically effective amount of both dexamethasone and promethazine. Where more than one patch is contained within the kit, at least one patch contains dexamethasone and a second patch comprises promethazine. The combination of dexamethasone and promethazine in the two patches is therapeutically effective. In some embodiments, the first patch contains dexamethasone and the second patch contains promethazine. In particular embodiments, the first patch is substantially free of promethazine and the second patch is substantially free of dexamethasone. In other embodiments, one or both of the patches may contain a combination of promethazine and dexamethasone, so long as the combined dose in the two patches is a therapeutically effective combination of dexamethasone and promethazine. The kit may comprise multiple patches representing multiple doses. For example, the kit may comprise sufficient number of patches to provide 1 to about 30 days' worth of the combination of dexamethasone and promethazine, preferably 1 to about 10 days' worth, particularly about 1 to about 7 days' worth.

The foregoing and further characteristics and advantages of the present invention are further described by the following detailed description of the invention.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The invention meets the foregoing need and provides further advantages as well by providing a pharmaceutical composition for transdermal drug delivery, comprising dexamethasone and promethazine, or a pharmaceutically acceptable salt thereof. The composition is adapted to deliver a combination of dexamethasone and promethazine across the skin. In some embodiments, the composition provides a therapeutically effective dose of a combination of dexamethasone and promethazine. In some particular embodiments, the composition is presented in unit dosage form. In some embodiments, the composition comprises an anti-emetic and/or anti-nausea effective amount of the combination of dexamethasone and promethazine, or a pharmaceutically acceptable salt thereof. In some embodiments, the composition further comprises a skin penetration enhancer.

Dexamethasone

Dexamethasone [(11β,16α)-9-fluoro-11,17,21-trihydroxy-16-methylpregna-1,4-diene-3,20-dione] is a corticosteroid with the structural formula:

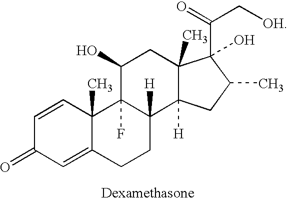

Dexamethasone is a known corticosteroid, having anti-inflammatory and immune-suppressing activity. The present invention provides transdermal compositions containing an effective amount of a combination of dexamethasone and promethazine, optionally in admixture with one or more skin penetration enhancers, such as dimethylsulfoxide (DMSO). The present invention further provides a method of treating or preventing nausea or emesis in a patient in need of such therapy, comprising administering via a transdermal route of administration a therapeutically effective amount of a combination of dexamethasone and promethazine to the patient. In some embodiments, the transdermal route of administration comprises applying a patch capable of administering a therapeutically effective amount of a combination of dexamethasone and promethazine to the patient. In other embodiments, the transdermal route of administration comprises applying to a patient's skin a therapeutically effective amount of a transdermal solution or suspension, such as an ointment, cream, gel or lotion, comprising a therapeutically effective combination of dexamethasone and promethazine, and optionally comprising a skin penetration enhancer.

Promethazine

Promethazine (N,N,α-trimethyl-10H-phenothiazine-10-ethanaamine) is an antihistaminic compound having the formula:

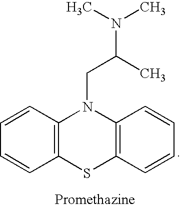

Promethazine is an antihistamine known to possess anti-allergy as well as antiemetic properties. In particular, promethazine has been used to treat motion sickness as well as post-operative (anesthesia-induced) nausea and vomiting. The present invention provides transdermal compositions containing an effective amount of a combination of dexamethasone and promethazine, optionally in admixture with one or more skin penetration enhancers, such as dimethylsulfoxide (DMSO). The present invention further provides a method of treating or preventing nausea or emesis in a patient in need of such therapy, comprising administering via a transdermal route of administration a therapeutically effective amount of a combination of dexamethasone and promethazine to the patient. In some embodiments, the transdermal route of administration comprises applying a patch capable of administering a therapeutically effective amount of a combination of dexamethasone and promethazine to the patient. In other embodiments, the transdermal route of administration comprises applying to a patient's skin a therapeutically effective amount of a transdermal solution or suspension, such as an ointment, cream, gel or lotion, comprising a therapeutically effective combination of dexamethasone and promethazine, and optionally comprising a skin penetration enhancer.

The foregoing and other needs are met by embodiments of the invention that provide a composition for transdermal drug delivery, comprising dexamethasone and promethazine, or a pharmaceutically acceptable salt thereof, in admixture with a pharmaceutically acceptable transdermal delivery vehicle. In some embodiments, the composition is in unit dosage form. In some particular embodiments, the composition comprises an anti-emetic and/or anti-nausea effective amount of the combination of dexamethasone and promethazine, or a pharmaceutically acceptable salt thereof. In some embodiments, the composition further comprises a skin penetration enhancer.

Further, the foregoing and other needs are met by embodiments of the invention that provide a composition for transdermal drug delivery, comprising dexamethasone and promethazine, or a pharmaceutically acceptable salt thereof, in admixture with a pharmaceutically acceptable transdermal delivery excipient, adjuvant or penetration enhancer. In some embodiments, the composition is in unit dosage form. In some particular embodiments, the composition comprises an anti-emetic and/or anti-nausea effective amount of the combination of dexamethasone and promethazine, or a pharmaceutically acceptable salt thereof. Furthermore, the invention provides a transdermal patch comprising a therapeutically effective amount of dexamethasone and promethazine, or a pharmaceutically acceptable salt thereof. In some embodiments, the patch is selected from a single layer medicine in adhesive patch, a multi-layer medicine in adhesive patch, a reservoir patch, a matrix patch, a microneedle patch or an iontophoretic patch.

The present invention also provides, in certain embodiments, a method of treating or preventing emesis in a subject, comprising transdermally administering to the subject a therapeutically effective amount of a combination of dexamethasone and promethazine or a pharmaceutically acceptable salt thereof. In some embodiments, the method comprises employing a therapeutically effective amount of dexamethasone and promethazine is administered in admixture with a topically-applied vehicle. In other embodiments, the method comprises using a therapeutically effective amount of dexamethasone and promethazine is administered as a transdermal patch. In still further embodiments, the transdermal patch is adapted for sustained release.

Transdermal Administration of Dexamethasone and Promethazine

The present invention provides for the transdermal administration of a therapeutically effective amount of a combination of dexamethasone and promethazine. The phrase “Therapeutically effective” means that amount of the combination of dexamethasone and promethazine that is effective to treat or prevent nausea and/or vomiting. In this context, “to treat” nausea or vomiting means to reduce the intensity of nausea and/or vomiting, and includes complete alleviation of nausea or vomiting for a period of time. In the context of this invention, “to prevent” means to protect the patient from the onset of nausea and/or vomiting for a period of time. In some embodiments, the period of time for which the treatment or prevention is provided is from about 30 minutes to about 48 hours, especially about 1 hour to about 24 hour. In some specific embodiments, prevention or treatment is provided for about 3 hours, about 4 hours, about 6 hours, about 8 hours, about 12 hours, about 18 hours, about 24 hours, about 48 hours or about 72 hours. In other embodiments, e.g. where prevention or treatment is provided for acute onset nausea and/or vomiting, prevention or treatment may be for an indefinite time period.

As used herein, all percent values relating to dexamethasone and promethazine are in units of % (weight of active per volume of solution, suspension or the like), unless otherwise specified.

In the context of the present invention, the term “transdermal delivery” means delivery of the combination of dexamethasone and promethazine across the skin and into the bloodstream. Thus, in the context of the present invention, transdermal administration results in systemic administration of the combination of dexamethasone and promethazine, especially systemic administration that avoids the portal vein of the liver, thereby avoiding first-pass effects associated with dexamethasone and/or promethazine.

Transdermal Patches

Transdermal patches (also referred to as skin patches) are medicated adhesive patches designed to provide medication to a patient. Some advantages of transdermal release include simplicity of use and concomitantly increased patient compliance, avoidance of the portal vein and resulting avoidance of the first-pass hepatic metabolism of the drug due, and in some cases sustained release of the drug over time. In addition, each type of transdermal patch presents other advantages specific to that type of patch.

Various transdermal patches are known in the art and considered suitable for practicing the present invention. The oldest known type of transdermal patches are the single layer patches, which comprise a patch substrate (or backing) and an adhesive, the latter of which contains the medication to be released into the patient. The patch is adhered to a detachable liner during storage; and the liner is removed and discarded prior to use. A layer of medication-containing adhesive is applied in one or more layers onto the substrate. The adhesive is adhered to the user's skin during use. Medication is released directly from the adhesive and through the skin into the patient's body. Thus, in some embodiments the invention provides a single layer transdermal adhesive patch, which comprises an adhesive in which the combination of dexamethasone and promethazine is dissolved. In some embodiments, the adhesive layer further comprises one or more penetration enhancers, which enhance the ability of dexamethasone, promethazine or both to penetrate the skin.

Another type of known patch is the multiple-layered medication in adhesive patch. It too comprises a substrate and an adhesive that contains the medication. However, rather than being applied in a single layer, the adhesive is applied in multiple layers. In some cases, the multiple-layered patch further comprises a membrane that separates the multiple layers. In other cases, the membrane is omitted. In some cases, the membrane provides additional mechanical support for the additional layers of medicated adhesive. In other cases, the membrane provides a dampening effect on the release of at least some of the medication from the patch and into the body. In any case, the medication is mixed into the adhesive, from which it is released once the patch has been applied to the body. The layer of adhesive farthest from the substrate is adhered to the patient's body. Thus, in embodiments of the invention, there is provided a multiple-layered medication patch in which dexamethasone is provided in at least a first layer of the patch and promethazine is contained within at least a second layer of the patch. In some embodiments, at least some of the pharmaceutically effective amount of dexamethasone and promethazine is contained within the first layer. In some more particular embodiments, at least some of the pharmaceutically effective amount of dexamethasone and promethazine is contained within the second layer. In more particular embodiments of the invention, at least some of the pharmaceutically effective amount of dexamethasone and promethazine is contained within the first and second layers. In even more particular embodiments of the invention, the pharmaceutically effective amount of each of dexamethasone and promethazine is substantially evenly divided between the first and second layers. In some embodiments, the first layer, the second layer or both further comprise one or more penetration enhancers, which enhance the ability of dexamethasone, promethazine or both to penetrate the skin.

A third type of transdermal patch is the reservoir type patch, which comprises a substrate, a reservoir, a medication-containing solution within the reservoir and an adhesive applied to the skin-facing side of the patch. In some embodiments, therefore, the invention provides a reservoir patch comprising dexamethasone and promethazine in the reservoir. In some embodiments, the reservoir further contains a skin penetration enhancer, which increases the ability of dexamethasone, promethazine or both to cross the skin.

Matrix patches comprise a medication contained within a matrix, which is a semisolid in which the medication is dissolved or suspended. The adhesive is applied to the skin-facing side of the matrix. Thus, embodiments of the invention comprise a matrix patch in which a therapeutically effective amount of dexamethasone and promethazine are dissolved or suspended in the matrix. In some embodiments, the matrix also contains one or more skin penetration enhancers, which enhance the ability of dexamethasone, promethazine or both to cross the skin.

Micro-needle patches are transdermal patches having micro- or nano-scale needles that pierce the cornified outer layer of the skin, thereby allowing the medication to pass from a reservoir or matrix and into the skin. Thus, embodiments of the invention comprise a microneedle patch comprising, within a reservoir or matrix, a therapeutically effective quantity of dexamethasone and promethazine.

Iontophoretic patches work by applying a current to the drug, causing the drug to move out of the adhesive, matrix or reservoir and through the skin. As iontophoresis requires that the drug species be in an ionic form, one embodiment of the invention provides a transdermal patch comprising a therapeutically effective amount of pharmaceutically acceptable salts of dexamethasone and a promethazine in a matrix, adhesive or reservoir. The patch also comprises means for applying an electrical charge to the salts sufficient to force the drug combination out of the patch and through the skin. Means for applying an electrical current can include batteries, capacitors, and the like.

In some embodiments, the present invention provides transdermal patches for delivery of a therapeutically effective amount of dexamethasone and promethazine. Transdermal patches have the advantage of providing controlled delivery of a dexamethasone and promethazine systemically, by delivering dexamethasone and promethazine across the skin and into the bloodstream. In some embodiments, dexamethasone and promethazine can be dissolved, dispersed or otherwise integrated into a suitable medium, such as an elastomeric matrix material. In some embodiments, penetration enhancers are used to increase the flux of the compound across the skin. The rate of such flux can be controlled by either providing a rate-controlling membrane or dispersing the compound in a polymer matrix or gel.

A variety of types of transdermal patches are employed in embodiments of the invention. In some embodiments, an adhesive patch or adhesive matrix patch is prepared from a backing material and an adhesive, such as an acrylate adhesive. In such embodiments, dexamethasone and promethazine, and optionally a penetration enhancer, are formulated into an adhesive casting solution and allowed to mix thoroughly. The solution is then cast directly onto the backing material and the casting solvent is evaporated in an oven, leaving an adhesive film. A release liner is then attached to complete the system.

In some embodiments, the invention provides a polyurethane matrix patch, which is employed to deliver the combination of dexamethasone and promethazine. The layers of this patch comprise a backing, a polyurethane drug/enhancer matrix, a membrane, an adhesive, and a release liner. The polyurethane matrix is prepared using a room temperature curing polyurethane prepolymer. Addition of water, alcohol, and the combination of dexamethasone and promethazine to the prepolymer results in the formation of a tacky firm elastomer that can be directly cast onto the backing material. A further embodiment of this invention uses a hydrogel matrix patch. In some embodiments, the hydrogel matrix comprises alcohol, water, a combination of dexamethasone and promethazine and hydrophilic polymers. The hydrogel matrix is then incorporated into a transdermal patch between the backing and the adhesive layer.

In some embodiments, the invention provides foam matrix patches, which are similar in design and components to the liquid reservoir system, except that the gelled drug solution is constrained in a thin foam layer, typically a polyurethane. This foam layer is situated between the backing and the membrane which have been heat sealed at the periphery of the patch.

In some embodiments, the invention provides a liquid reservoir patch comprising dexamethasone and promethazine. This type of patch comprises an impermeable or semi-permeable, heat-sealable backing material, a heat sealable membrane, an acrylate based pressure sensitive skin adhesive and a siliconized release liner. The backing is heat sealed to the membrane to form a reservoir, which is filled with a solution of the combination of dexamethasone and promethazine, penetration enhancer(s), gelling agent, and other excipients.

In some embodiments, the invention provides a transdermal patch containing about 0.5 mg to about 100 mg of dexamethasone and about 1 mg to about 500 mg, especially about 5 mg to about 250 mg, and more particularly about 10 mg to about 100 mg, of promethazine per patch. In some embodiments, a patch adapted to release its contents into the patient over a period of about 12 hours releases about 0.5 mg to about 20 mg of dexamethasone and about 5 mg to about 100 mg, especially about 5 mg to about 50 mg, of promethazine into the patient's system per 12 hour period. In some embodiments, in which the patch is adapted to release its contents over a period of 3 to 6 hours, the patch contains about 0.5 mg to about 40 mg of dexamethasone and about 5 mg to about 100 mg of promethazine, especially about 10 mg to about 50 mg of promethazine. In some embodiments the patches according to the invention provide about 30 minutes to about 72 hours, more particularly about 1 hour to about 24 hours, of prevention or treatment of nausea and/or vomiting per patch.

Transdermal Solutions, Suspensions and the Like

In some embodiments, the invention provides a transdermal ointment, cream, gel, lotion or other transdermal solution or suspension. The transdermal ointment, cream, gel or lotion is made by admixing a suitable quantity of dexamethasone and promethazine with one or more excipients for the preparation of the ointment, cream, gel or lotion, in a conventional manner. In some particular embodiments, the transdermal composition further comprises at least one penetration enhancer, such as dimethyl sulfoxide, for improving the permeability of the skin and allowing passage of dexamethasone and promethazine through the skin and into the bloodstream of the patient.

In some embodiments, a unit dose of the combination of dexamethasone and promethazine is in the range of about 50 μl to about 1,000 μl, especially about 100 to about 500 μl per dosage unit. In some embodiments, a unit dose of the combination of dexamethasone and promethazine contains about 0.5 mg to about 20 mg of dexamethasone and about 1 mg to about 500 mg, especially about 5 mg to about 250 mg, more particularly about 10 mg to about 100 mg of promethazine. In some embodiments, a dosage unit of a composition according to the invention contains about 0.5 mg to about 100 mg of dexamethasone and about 5 mg to about 50 mg, especially about 5 mg to about 50 mg of promethazine per unit. Thus, in some embodiments, the transdermal solution or suspension of the invention contains about 0.1% (weight/volume) to about 5% (especially about 0.5% to about 4%, and most especially about 1% to about 2%) dexamethasone and about 0.1% (weight/volume) to about 10% (weight/volume) of promethazine. In addition to conventional ointment, cream, gel and lotion ingredients, some embodiments of the invention include one or more penetration enhancers to aid in the penetration of dexamethasone, promethazine or both through the skin and into the bloodstream.

The lotion, ointment, gel or cream should be thoroughly rubbed into the skin so that no excess is plainly visible, and the skin should not be washed in that region until most of the transdermal penetration has occurred preferably at least about 15 minutes and, more preferably, at least about 30 minutes. In some embodiments, each unit dose of transdermal solution, suspension or the like contains about 0.5 mg to about 20 mg of dexamethasone per dose and about 10 mg to about 100 mg of promethazine per dose and is administered about once every 3 to 24 hours, especially about once every 6 to 24 hours, and more particularly about 3 to 4 times per day.

Antiemetic Kits

The antiemetic compositions according to the invention are, in some embodiments, presented as part of a kit for the treatment or prevention of emesis and/or nausea. In some preferred embodiments, the kits comprise instructions for the antiemetic use of the compositions of the invention. In some embodiments, the kits comprise two or more transdermal patches comprising a therapeutically effective amount of dexamethasone and promethazine. In some particular embodiments, the kits comprise at least a first patch comprising dexamethasone and at least a second patch comprising promethazine. In some preferred embodiments, the first patch is substantially free of promethazine and/or the second patch is substantially free of dexamethasone. In such embodiments, the combination of dexamethasone in the first patch and promethazine in the second patch is a therapeutically effective amount of the combination of dexamethasone and promethazine. In such embodiments, the first and second patches constitute one dosage unit. In other embodiments, the invention provides at least a single patch comprising a therapeutically effective amount of a combination of dexamethasone and promethazine. In such embodiments, the single patch constitutes one dosage unit. Thus, a dosage unit is made up of sufficient dexamethasone and promethazine to constitute a therapeutically effective dose of the combination of dexamethasone and promethazine, whether divided between two or more patches or combined in a single patch.

In some embodiments, the kit comprises more than one dosage unit. In particular embodiments, the kit comprises from 1 to about 120 or more, from 1 to about 60, from 1 to about 30, from 1 to about 10 or from 1 to about 7 dosage units. In cases where the patch(es) is (are) adapted to release a therapeutically effective amount of dexamethasone and promethazine over a 24 hour period, the kit conveniently comprises 1, about 5, about 7, about 10, about 14 or about 30 dosage units. In cases where the patch(es) is (are) adapted to provide a therapeutically effective amount of dexamethasone and promethazine over a 12 hour period, the kit conveniently comprises 1, 2, about 10, about 14, about 30 or about 60 dosage units. In cases where the patch(es) is (are) adapted to provide a therapeutically effective amount of dexamethasone and promethazine over an about 3 to about 10 hour (especially an about a 6 or 8 hour) period, the kit comprises about 1, about 4, about 40, about 60 or about 120 dosage units. The person skilled in the art will recognize that other numbers of dosage units may be included in the kit without departing materially from the present invention.

Although preferred embodiments of the present invention have been shown and described herein, it will be apparent to those skilled in the art that such embodiments are provided by way of example only. Numerous variations, changes, and substitutions will be apparent to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered herein.

What is claimed is:

1. A composition for transdermal drug delivery, comprising dexamethasone and promethazine, or a pharmaceutically acceptable salt thereof.

2. The composition of claim 1 in unit dosage form.

3. The composition of claim 1, comprising an antiemetic amount of the combination of dexamethasone and promethazine, or a pharmaceutically acceptable salt thereof.

4. The composition of claim 1, further comprising a skin penetration enhancer.

5. A composition for transdermal drug delivery, comprising dexamethasone and promethazine, or a pharmaceutically acceptable salt thereof, in admixture with a pharmaceutically acceptable transdermal delivery vehicle.

6. The composition of claim 5 in unit dosage form.

7. The composition of claim 5, comprising an antiemetic amount of the combination of dexamethasone and promethazine, or a pharmaceutically acceptable salt thereof.

8. The composition of claim 5, comprising a skin penetration enhancer.

9. A composition for transdermal drug delivery, comprising dexamethasone and promethazine, or a pharmaceutically acceptable salt thereof, in admixture with a pharmaceutically acceptable transdermal delivery excipient, adjuvant or penetration enhancer.

10. The composition of claim 9 in unit dosage form.

11. The composition of claim 9, comprising an antiemetic amount of the combination of dexamethasone and promethazine.

12. A transdermal patch for the treatment or prevention of nausea and/or emesis in a subject, comprising a therapeutically effective amount of dexamethasone and promethazine, or a pharmaceutically acceptable salt thereof.

13. The transdermal patch of claim 12, wherein the patch is a single layer medicine in adhesive patch.

14. The transdermal patch of claim 12, wherein the patch is a multi-layer medicine in adhesive patch.

15. The transdermal patch of claim 12, wherein the patch is a reservoir patch.

16. The transdermal patch of claim 12, wherein the patch is a matrix patch.

17. The transdermal patch of claim 12, wherein the patch is a microneedle patch.

18. The transdermal patch of claim 12, wherein the patch is an iontophoretic patch.

19. The transdermal patch as in any one of claims 12-18, further comprising a skin penetration enhancer.

20. A method of treating or preventing nausea and/or emesis in a subject, comprising transdermally administering to the subject a therapeutically effective amount of a combination of dexamethasone and promethazine or a pharmaceutically acceptable salt thereof.

21. The method of claim 20, wherein the therapeutically effective amount of dexamethasone and promethazine is administered in admixture with a topically-applied vehicle.

22. The method of claim 20, wherein the therapeutically effective amount of dexamethasone and promethazine is administered as a transdermal patch.

23. The method of claim 22, wherein the transdermal patch is adapted for sustained release.

24. A kit comprising an antiemetically effective combination of dexamethasone and promethazine for transdermal administration.

25. The kit of claim 24, comprising a first patch comprising dexamethasone.

26. The kit of claim 25, comprising a second patch comprising promethazine.

27. The kit of claim 26, wherein the second patch is substantially free of dexamethasone.

28. The kit of claim 26, wherein the first patch is substantially free of promethazine.

29. The kit of claim 24, comprising a single patch comprising the combination of dexamethasone and promethazine.

30. The kit as in any one of claims 24-29, further comprising instructions for the transdermal, antiemetic use of the antiemetically effective combination of dexamethasone and promethazine.
